(19) United States
(12) Patent Application Publication
(10) Pub. No.: US 2007/0148437 A1
(43) Pub. Date: Jun. 28, 2007

(54) Thermosensitive, biocompatible polymer carriers with changeable physical structure for therapy, diagnostics and analytics

(75) Inventor: Detlef Müller-Schulte, Aachen (DE)

Correspondence Address: DAVIDSON, DAVIDSON & KAPPEL, LLC, 485 SEVENTH AVENUE, 14TH FLOOR, NEW YORK, NY, 10018, US

(73) Assignee: MagnaMedics GmbH, Aachen (DE)

(21) Appl. No.: 10/578,024

(22) PCT Filed: Oct. 22, 2004
(86) PCT No.: PCT/EP04/11937

§ 371 Date: Apr. 28, 2006

(30) Foreign Application Priority Data
Oct. 28, 2003 (DE) 10350248.3

(51) Int. Cl. A61K 8/72; B32B 5/16; C08K 9/00

(52) U.S. Cl. 428/327; 428/402; 428/692.1; 428/403; 523/105; 523/200; 523/210

(57) ABSTRACT

The invention relates to biocompatible, thermosensitive polymers that can be heated, with the aid of a high-frequency magnetic alternating field, by encapsulating magnetic and/or metallic colloids or magnetic nanoparticles. As a result of the inductive heating of the polymer matrix, physical structure changes are triggered in the polymer matrix that lead to the bioactive substances encapsulated in the matrix being released within a short period of time. This “stimulus-response” principle is employed for the production of controllable drug depots, contrast-intensifying media for NMR diagnostics or manipulable micro-tools, as media for blocking blood vessels and as controllable porogens in the production of membranes.

The invention relates to biocompatible and thermosensitive polymer carries of different particle sizes that enable in vivo application for therapeutical, diagnostic or analytical purposes. Encapsulated in the polymer matrix are substances in the form of magnetic and/or metallic colloids that can be heated by input of energy with the aid of a high-frequency magnetic alternating field, resulting in a change of the physical structure of the polymer matrix in the form of a change of configuration.

The invention also relates to the production and use of the polymer carriers.

Magnetic polymer particles that can be heated by induction are known from various publications and patents such as, for example, from the publication WO 03/101486 A2, which describes inductively heatable thermosensitive polymer carriers, based on N-isopropylacrylamide and acrylamide derivatives, which are caused to release encapsulated biosubstances or drugs on account of an inductive stimulus.

The isopropylamides described in the above-mentioned document are the most commonly used polymers with thermosensitive properties. At temperatures above 27° C. they experience a separation of phases which is accompanied by a shrinkage process. This shrinkage is reversible, i.e. if cooled to below 30° C. the polymer practically resumes its original shape. This special property of poly-N-isopropylacrylamide and the interesting applications that can be derived from this, for example as a drug depot, bio-sensor, cell culture substrate, cell encapsulation matrix, actuator or valve, have been known for a long time and are reflected in a number of publications and patents.

The disadvantage of the media described in the above documents consists in the use of acrylamide derivatives. This class of compounds is not unobjectionable as regards bio-compatibility since it cannot be excluded that after polymerization, monomers or oligomers that have not been completely converted still remain in the polymer. These monomers are considered as highly toxic, and in the case of a possible in vivo application they can diffuse out of the polymer. For these reasons, active substance carriers made from these polymers can only be used for in vivo applications to a very limited degree.

The application WO 03/101486 A2 at the same time represents the nearest prior art and offers a good overview thereof. A common feature of all the further media and processes cited in that document is that without exception the magnetic induction serves to heat the particles or to destroy cells or biological organisms by over-heating, or that that their function is derived exclusively from the complementary interaction of a bioligand or bioreceptor, bound on the matrix, with the substance to be analyzed.

Their fields of use are thus restricted to the known fields of tumor treatment and the separation and analysis of bio-molecules or the marking of certain cells using the classical principle of affinity. They thus differ from the media according to the invention in that on account of their chemical structure they are not thermosensitive, i.e. they are not able to change their physical structure or geometric shape on the basis of an internal thermal stimulus caused by external induction.

This property, however, is the basic condition for using polymer carriers as manipulable or controllable micro- or nanocarriers and/or micro- or nanotools.

Edelmann et al., J. Biomed. Mat. Res., Vol. 21, 339, 1987, describe an ethylene-vinyl acetate copolymer which contains magnets embedded therein that are of a size in the millimeter range. Encapsulated serum albumin is released by means of an oscillating magnetic field. As the polymer and the encapsulated magnet are relatively large macroscopic bodies which can only be applied by implantation and are thereafter not automatically discharged by the body, this method is excluded from a large number of in vivo applications.

In summary, it may be said that all of the media or products described in the prior art have in common that they are either not unobjectionable as regards biocompatibility or that wherever these are non-magnetic polymer carriers, a change in the physical structure or shape can only be triggered through heat that is applied directly from the outside, and that wherever they are magnetic carriers, their structure cannot be changed in any way, neither through an external stimulus nor through externally applied energy. Furthermore, the “stimulus-response” carriers known from the state-of-the-art are either irregular nano-particles or larger volume mass polymers that are unsuitable as carriers for active agents (drugs), as a contrast medium in NMR diagnostics (magnetic resonance tomograph), as media for molecular separation or as controllable micro-tools for in vivo applications.

Object

The object of the present invention is to produce thermosensitive and biocompatible polymer carriers which stand out for their good biocompatibility or biodegradability and which can be selectively stimulated by an energy supply, e.g. in the form of magnetic induction, to induce a change in the physical structure of the polymer matrix on the basis of the resulting increase in temperature. The polymers are preferably spherical, nano- or microparticulate particles or fibers, tubes or threads. Since the change in the configuration of the polymers of the present invention is in the range from 27-50° C., and thus in the range of the body temperature (37° C.), these carriers can be used in vivo.

Through the use of inductive heating, dosing systems for the administration and application of active agents can be created for the medical field or analytics that are characterized in particular by their contact-free controllability. An active substance/active agent is understood as meaning a substance that triggers a chemical, biochemical or physiological reaction in one way or another and hereby creates a therapeutic, diagnostic and/or prophylactic effect or can fulfill an analytical function. Examples include biologically active proteins or peptides, enzymes, antibodies, antigens, nucleic acids, glycoproteins, lectins, oligosaccharides, hormones, lipids, growth factors, interleukins, cytokines, steroids, vaccines, anticoagulants, cytostatic agents, immunomodulatory agents or antibiotics. To this end, the active agents are encapsulated in the polymer particles. As a rule, this is carried out by a direct admixing of the corresponding active agent to the soluble polymer phase.

The carriers charged with the corresponding active agent that are produced in this way can then be applied to the desired physiological or bio-analytical sites of action with the aid of known administration methods such as injection, implantation, infiltration, diffusion, streaming or biopsy. The local application of the magnetic particles can be further intensified by positioning the particles exactly at the desired spots using electro- or strong permanent magnets that are placed over the target area or site of action from the outside. Once the polymer particles have reached their site of action they can be heated up to above body temperature by applying a high-frequency magnetic alternating field, resulting in a change in the physical structure of the polymer matrix. In accordance with the invention, a “change in the physical structure” is hereby defined as a change in the original molecule configuration, including the conformation on account of a swelling or de-swelling process, leading to a change in the geometric shape, volume or particle size of the polymer carrier. The change in volume may be manifested for example in a shrinkage or swelling process with a parallel change in the pore size, or in a change of the external shape (geometry) of the polymers. Changes in the physical structure can also mean that the molecular configuration returns to its original form that has been temporarily changed through a heating and cooling process (freezing process) (“shape-memory-polymer”).

The change in the physical structure therefore triggers a concentrated and quick release of the encapsulated active agents from the matrix.

The time that is needed for the active agent to diffuse out from the gel is basically dependent on the size of the polymer carrier (micro- or nanoscale) as well as on its molar mass, the molar mass of the active substance, and on the temperature generated in the polymer capsule. Nanoscale polymer particles generally have a release time that is, by a factor of 0.1 to 0.5, shorter than that of corresponding microgels (>20 μm), under otherwise analogous conditions (same active agent, temperature, polymer).

It is thus a subject matter of the present invention to make the polymers useful as carriers for therapeutic, analytic or diagnostic applications and to encapsulate active agents or drugs in the polymer carriers and after corresponding in vivo administration to apply these selectively and controllably with the aid of magnetic induction or to move them to the site of action using a magnet. The object of the invention of selectively releasing the encapsulated active substances or drugs is solved by heating the thermo-sensitive polymers through magnetic induction, i.e. through an externally applied, high-frequency magnetic alternating field, by encapsulating magnetic and/or metallic substances in the polymer matrix that are able to absorb energy from the magnetic field and can thereby heat up the polymer carrier to above the body temperature.

A further subject matter of the invention is the production of polymers that shrink at temperatures above the body temperature, so that the polymer carriers, at temperatures above the body temperature, are applied in a deswollen (shrunk) state and return to a swollen state after cooling to body temperature. This phenomenon can be applied in the context of therapeutic anti-tumor measures. One of the fatal pathological developments during tumor development is angiogenesis. This is generally understood as being a great dissemination in the formation of blood vessels in the tumor tissue. This pathological process, which up to now has been primarily treated with drugs (or by operations), can now be surprisingly suppressed or greatly delayed with the aid of the media in accordance with the invention. To this end, particles, preferably with a particle size of 0.3 μm to 5 μm, that have been heated in advance to temperatures >45° C. and have thus reached their maximum degree of shrinkage, are introduced into the tumor tissue. As a result of the subsequent adaptation to the body temperature the particles start to swell again and reach their equilibrium swelling status after a few minutes. In this swollen state the polymer carriers have an embolising function, i.e. they are able to block the blood vessels and thus combat the development of tumors. This special property is displayed in particular by polymers such as, for example, hydroxyalkyl cellulose, isopropyl cellulose, polyoxyethylene, and poly(ethylene glycol-lactide-glycolide) copolymers. Particles with a particularly wide range of sizes (0.3 to 10 μm) are suitable to combat angiogenesis in practice since they allow blood vessels of all widths to be blocked at once.

On account of the special combination of product and process, the polymer carriers in accordance with the invention can thus be used in particular as a matrix for the encapsulation of active agents and as media for blocking blood vessels.

The combination of heating induced by the magnetic field of biocompatible, thermosensitive polymer carriers with a parallel change in the physical structure and/or carrier geometry thus creates a range of properties that go far beyond former polymer carrier systems.

The specific application of the polymer carriers in accordance with the invention in conjunction with an externally controllable structural change surprisingly opens up the possibility of exploiting new integral active combinations.

It is thus a further object of the present invention to produce polymer particles as a new type of contrast-intensifying medium in the context of NMR diagnostics and parallel to this as a basis for a controllable application of active agents. From the state of the art already discussed it is known that superparamagnetic, ferromagnetic or paramagnetic substances can lead to a substantial intensification of the contrast during imaging in the context of NMR diagnostics (e.g. magnetic resonance tomography, MRT). The receptor-specific coupling of bioligands to the media of the invention enables a more precise diagnosis through a better localization and classification of pathological processes (e.g. detection of tumors in early stages and micro-metastases).

In order to heat the aforementioned magnetic and metallic substances and compounds up to the temperatures that are relevant for the analytical, therapeutic and diagnostic applications, a special design of the magnetic field is required with respect to the magnetic field strength and frequency. Commercially available systems consisting of current-carrying coils are normally used that are fed from a high-frequency generator. The coil dimensions depend on the sizes of the respective samples and on the area to be irradiated; they are generally 5 to 30 cm in diameter and 5-30 cm long. The necessary output of the HF generators is normally between 1.5 and 4.5 kW. Two generator settings can in principle be used to heat up the magnetic samples: a) a high frequency in the range 5-20 MHz with a low magnetic field strength of 100-500 A/m or b) a low frequency of 0.2-0.8 MHz in combination with a high field strength of 1 to 45 kA/m. Both field parameter combinations guarantee a sufficient thermal output within a short application period (<1 min.). Sufficient energy to heat up the carriers can also be provided with larger coil geometries (30-40 cm diameter) by a corresponding increase in the field strength to >15 kA/m for the radiation of regions with a larger volume, as is the case for example in the application of medical active agents in certain parts of the body.

The starting point for the synthesis of thermosensitive polymer carriers are magnetic colloids in the form of ferromagnetic, ferrimagnetic or superparamagnetic nanoparticles or microparticles that display a high magnetization and can be inductively heated in a magnetic alternating field and have a Curie temperature in the range of from 30° C. to 100° C. The preferred substance for this purpose is magnetite (Fe₃O₄) or γ-Fe₂O₃. The production of such colloids is sufficiently described in the literature. Iron (III) and iron (II) saline solutions with varying molar ratios (0.5:1, 2:1 to 4:1) always form the basis to produce magnetite or γ-Fe₂O₃, these then being converted into corresponding colloidal magnetic dispersions (“magnetic colloids”) by adding bases or applying heat. In order to prevent an agglomeration of the fine magnetic particles, which is caused, in particular, by the van der Waals' forces, surface active agents (tensides, emulsifiers and stabilizers) can be added that practically prevent a precipitation of the colloid in an aqueous dispersion. Such stabilized colloidal dispersions are also known under the name “ferrofluids” and are commercially available (Ferrofluidics Corp., USA; Advanced Magnetics, USA; Taibo Co, Japan; Liquids Research Ltd., Wales; Schering AG, Germany).

The surface-active substances used are cationic, anionic or non-ionic, such as, for instance: oleic acid, lauryl sulfonate, phosphate ester, alcohol ether sulfates, alkylaryl polyether sulfates, alkylaryl polyether sulfonates, citrates, alkyl naphthalene sulfonates, polystyrene sulfonic acid, polyacrylic acid or petroleum sulfonates (anionic tensides), dodecyl trimethylammonium chloride (cationic tenside) and nonyl phenoxypolyglycidol, polyvinyl alcohol, kerosene, alkylaryl oxypolyethoxy ethanols, nonylphenol or polyethylene glycols (non-ionic tensides).

The particle sizes of the magnetic colloids depend, as is generally known, on various test parameters such as the iron salt ratio, base concentrations, pH value and temperature.

The magnetic colloids suitable for the media in accordance with the invention all have a particle size of 5-800 nm, preferably one of 10-500 nm. This guarantees that the magnetic colloids are present in a colloidally dispersed form during subsequent encapsulation in the polymer matrix. Through a selective, metered addition of corresponding amounts of the appropriate colloid, the magnetic properties and as a consequence the heat-up properties of the polymer carrier can be specifically controlled.

The concentrations of magnetic colloids in the monomer or polymer formulation are normally 10 to 40% by volume, in which case the solid content of the magnetic substance relative to the polymer phase is generally 5 to 40% by weight, preferably 10 to 30%.

Apart from magnetic colloids, metallic colloids can also be encapsulated in the polymer matrix as an alternative. All metallic substances in a colloidal or finely dispersed form that can be inductively heated in a high-frequency alternating field are in principle suitable. Since physiological applications of the media in accordance with the invention represent an essential aspect, those metal colloids that can be inductively heated which are physiologically harmless and/or chemically-physically inert are preferably used. These include the metals in groups 8 to 11 (IUPAC definition 1986, i.e. Fe, Co, Ni, Cu and higher homologues), with gold, silver, palladium and platinum colloids or corresponding powders being preferably used on account of their biocompatibility.

The metal colloids used for the media in accordance with the invention normally have a particle size of between 5 and 200 nm. The production of such colloids, that have long been used to determine proteins and nucleic acids on account of their special absorption properties in the visible range in bioanalytics, above all the gold colloids, is sufficiently known from the state of the art, and a large number of metal colloids or metal powders are also available commercially. As any expert in this field knows, they are all produced through the reduction of the corresponding metallic salts or by the metal spraying method.

Both the metal colloids and corresponding powders can be used for the media and processes in accordance with the invention; these are admixed to the polymer formulation in the desired concentration before polymerization. The metal shares in the polymers and/or particles are normally between 5 and 40% by weight.

After adding the colloids it is often advantageous to briefly expose the magnetic colloid-polymer mixture to ultrasonic waves using an ultrasonic finger or ultrasonic bath to ensure a fine dispersion of the colloid. The fine-disperse distribution of the colloid enables a correspondingly better dissipation of heat in the polymer matrix, which in turn guarantees a continuous release of the encapsulated active agent.

As thermosensitive substances having high biocompatibility the following are used: polyethylene oxides, polylactides, polyglycolides, polysaccharides, polysaccharide derivatives, polyamino acids, polyethers, chitosan, polyvinyl alcohol, alginate, gelatin, or copolymers or block copolymers of these substances. To produce the thermosensitive polymers, the following processes are utilized depending on the type of polymer used:

a) ring-opening polymerization

b) suspension precipitation

c) suspension crosslinking processes

d) ionic crosslinking in suspension

e) salting-out emulsion processes

f) solvent evaporation processes

These processes according to the state of the art are generally known and will be explained in the following within the context of the polymers according to the invention. Microparticulate drug carriers based on poly(lactide) and poly(lactide-co-glycolide) can be produced with the known processes such as, for instance, solvent evaporation, phase separation or spraying processes or salting-out techniques. The basic principle of this approach is the use of water-soluble organic solvents, e.g. acetone, which are emulsified in an aqueous phase saturated with salt. The solvent evaporation process is preferably started from an aqueous solution of the encapsulated active substance, which is subsequently dispersed in the polymer phase. As an alternative, the active substances can also be dispersed directly in the polymer phase. Through subsequent addition of non-solvents—preferably vegetable or mineral oils—the polymer carrier is precipitated at the interphase. Acetone, benzene and methylene chloride are preferably used as solvents for the polymers, or chloroform for the poly(lactide-co-glycolide) copolymers. To improve the stabilization of the colloids, 0.1 to 1 mole percent (relative to the polymer) of proteins, such as serum albumin, or synthetic polymers, polyvinyl alcohol or polyvinyl pyrrolidone, may be added.

To synthesize the inventive magnetic carriers, 5 to 40% by volume, preferably 20-40%, of an organic-based ferrofluid is normally added to the polymer phase. Another advantage of the poly(lactide)-based polymers is their biodegradability over a period of months, the polymer being hydrolyzed within a matter of months and the hydrolysates subsequently being metabolized.

A further group of thermosensitive, biocompatible polymer carriers is derived from poly(ethylene glycol-lactide-ethylene glycol) block copolymers. These possess the special property of passing from a liquid state to a solid, gel-like state at temperatures above approx. 35° C. This phase transition can, surprisingly, be used to produce thermosensitive, magnetic nanoparticles, by dispersing magnetic nanoparticles or magnetic colloids, preferably having a particle size of between 5 and 100 nm, in a 10 to 30% aqueous solution of the polymer and then treating them with an ultrasound finger for 10 to 120 seconds at temperatures <15° C. This produces magnetic particles that are enveloped with the poly(ethylene glycol-lactide-ethylene glycol) block copolymer and form stable colloids. Due to their special gelling properties, these colloids can be used for treating tumors and metastases by injecting the colloids, which are low-viscous at room temperature, directly into the tumor tissue. Through the subsequent inductive heating of the colloid to above body temperature (>37° C.) the colloid is solidified to form a gel. As a consequence of the heat-induced gelation, the blood supplying vascular systems can be blocked and the further growth of the tumor suppressed.

Using the suspension precipitation method, further polymers can be produced that meet the criteria according to the present invention of biocompatibility and thermosensitivity. This group includes those polymers that are soluble in a particular solvent only at high temperatures and precipitate upon cooling of the solvent. Examples of polymer-solvent systems for producing the corresponding polymers that are suitable for this synthesis technology are (without limiting the invention in any way): polyvinyl alcohol-dimethyl formamide, polyvinyl alcohol-ethylene glycol, gelatin-water, agarose-water, cellulose tributyrate-ethanol, cellulose acetate butyrate-methanol, cellulose acetate butyrate-toluene, starch-water, cellulose-ZnCl₂ solution and collagen-water.

To arrive at spherical particles, the polymers, which are dissolved at higher temperatures, are dispersed in an organic phase that is immiscible with the polymer phase. Vegetable oils or mineral oils, which normally have a viscosity of between 40 and 400 cp, are suitable and preferred for this purpose. During the subsequent process of cooling down to room temperature, or temperatures <40°C., these polymers precipitate in the form of spherical particles.

By admixing magnetic colloids or ferrofluids, which form stable dispersions with the polymer phase, and active agents, e.g. in the form of zytostatic agents, magnetic active substance carriers are obtained.

The magnetic field-induced heating of these polymer carriers leads to a pronounced swelling process, which results in a concentrated release of the encapsulated drugs. The release kinetics is dependent both on the molar mass of the polymer and on the magnetic colloid content, the latter having a direct influence on the heating that may be achieved. Normally, the swellability increases as the molar mass decreases, and as a consequence the release rates of the incorporated active agents also increase.

To synthesize the polymer carriers using the suspension-precipitation process, 1 to 15% polymer solutions are used with preference. The concentration of the added magnetic colloid is generally 10 to 40% by volume, relative to the polymer phase. As incorporable active agents, those substances are suitable that form stable, homogenous, i.e. non-agglomerating, colloidal dispersions with the polymer phases. For example, plasmids, peptides, nucleic acids, oligosaccharides or zytostatic agents such as ifosfamide, melphalan, cyclophosphamide, chlorambucil, cis-platinum or methotrexate can be used for this purpose.

In the context of any of the inventive polymer carriers, it has been found that to improve the quality of the suspensions and particle geometries (spherical shape) it is advantageous to add at least one, but generally not more than three, surface-active substances to the oil phases. Examples of such additives, which do not restrict the invention, include: polyoxyethylene adducts, alkyl sulphosuccinates, polyoxyethylene sorbitol esters, polyethylene-propylene oxide block copolymers, alkyl phenoxypolyeth-oxyethanols, fatty alcohol glycol ether phosphoric esters, sorbitan fatty acid esters, sucrose stearate palmitate, fatty alcohol polyethylene glycol ether, polyglycerol esters, polyoxyethylene alcohols, polyoxyethylene sorbitan fatty acid esters and polyoxyethylene acids. Substances of this type are commercially available, inter alia, under the identifications Hostaphat, Isofol, Synperonic, Span, Tween, Brij, Aerosol OT, Hypermer, Myrj, Triton, Arlacel, Dehymuls, Eumulgin, Renex, Lameform, Pluronic or Tetronic. In order to control the size of the polymer drops formed in the suspension process, which should be (<1 μm), 0.05-15% by weight, preferably 0.5-5% by weight, of one or more tensides are preferably added to the oil phase.

The particle sizes of (<1 μm) are suitable above all for a biomedical in vivo application as they lastingly support the ability to penetrate tissue for such applications. Particles with a size of 20-200 nm are preferably used as contrast media in NMR diagnostics and as porogens to produce adjustable pore widths in membranes. Particles with a size between 200-800 nm, however, are utilized particularly as drug depots for the selective application of active agents, e.g. in the form of therapeutic, diagnostic or prophylactic agents.

The suspension process is normally carried out with a conventional stirrer or a dispersing machine. Propeller mixers with stirring speeds of between 600 and 1500 rpm are adequate for realizing particle sizes in the range from 10-500 μm; particle sizes <10 μm are normally realized by stirring speeds of >1500 rpm. On the other hand, only dispersing machines with stirring speeds of >5000 rpm are suitable for particle sizes of <1 μm. Stirrers that work according to the rotor-stator principle are used, above all, for this purpose. At these high mixing speeds it is preferable for the dispersions to be produced in an argon or nitrogen atmosphere or in a vacuum to largely rule out the introduction of air, which would affect the dispersion quality.

In a further process approach according to the invention, the use of oils as suspension medium can be dispensed with completely by starting from (meth)acrylate-substituted dextranes which are subsequently suspended in a polyethylene glycol phase. By varying the ratio of dextrane to polyethylene glycol, it is possible to vary the size of the polymer particles being formed, as is known from the state of the art (Stenekes et al. Pharm. Res., Vol. 15, 557, 1998). Normally, with polyethylene glycol/dextrane volume ratios of <40, the particle sizes are shifted towards larger particles (>10 μm). By further varying, inter alia, the degree of substitution, the acrylate substituents (e.g. hydroxyethyl methacrylate, glycidyl methacrylate) as well as the molar mass of the phases (polyethylene glycol, dextrane) employed, it is possible to obtain a wide range of different polymer carriers. After encapsulation of the corresponding magnetic colloids, as are described above, and encapsulation of certain active agents (e.g. peptides, plasmids), the carriers can be stimulated, with the aid of magnetic field-induced heating, within 5 minutes to release active agent in a controlled and concentrated manner.

A further group of biocompatible and thermosensitive carrier media in the sense of the invention is derived from the liposomes. Liposomes are synthetically produced spherical hollow bodies (vesicles) which are enveloped by a membrane consisting of a lipid layer or lipid double layer. They are biocompatible because the lipids constituting the membrane predominantly consist of components of natural cell membranes. On account of their vesicle structure, liposomes are particularly suitable for functioning as active substance carriers through encapsulation of drugs or other bioactive substances such as peptides or nucleic acids. It could be shown, surprisingly, that by encapsulating magnetic colloids or ferrofluids in the vesicles, as well as combining certain lipids, thermosensitive magnetic liposomes are formed which can be heated to temperatures above 37° C. using the above-explained magnetic field induction. Suitable lipids are in principle any natural lipids such as, for example, phosphatidyl choline, phosphatidylic acid, cholesterol, phosphatidyl ethanolamine, monosialogangliosides, phosphatidyl inosite, phosphatidyl serine and sphingomyelin. As is generally known, the lipid compositions can be varied, within certain limits, both with respect to the ratio among themselves and with respect to their concentration. Examples of such compositions are: Phosphatidyl choline:cholesterol:monosialogangliosides:2:1:0.14; sphingomyelin:monosialoganglioside 1:0.07; sphingomyelin:cholesterol:monosialogangliosides 2:1:0.13;sphingomyelin:phosphatidylcholine:cholesterol:1:1 1; phosphatidyl choline:cholesterol:phosphatidyl ethanolamine:1:1:0.2. Particularly by substituting with those lipids that stabilize the membrane conformation, such as sphingomyelin, it is possible to reduce phagocytosis by 90%. An analogous effect can also be achieved with polyethylene glycol (PEG)-substituted (pegylated) lipids, e.g. with PEG phosphatidyl ethanolamine. The molar ratios of the biocompatibility-increasing substituents to the other lipids are preferably between 0.1 and 0.4.

To produce magnetic liposomes, a dialysis process known from the state of the art (M. De Cuyper et al. Prog. Coll. Polym. Sci. Vol. 82, 353, 1990) is used. To this end, a ferrofluid, stabilized by lauric acid, is dialyzed in the presence of a unilamellar lipid vesicle. It could be shown, surprisingly, that by inductive heating of the magnetic liposomes to temperatures >45° C., the lamellar lipid conformation is changed in such a way that >60% of encapsulated active substances is released within 1 to 6 minutes. By heating further to >50° C., the vesicle structure is destroyed completely, so that the encapsulated active substances are completely released within one minute.

The thermosensitive and biocompatible media according to the invention also include the group of the polyoxyethylenes and polyoxypropylenes, as well as copolymers of these substances of the general formula HO—(CH₂—CH₂O)ₓ—(CH(CH₃)—CH₂O)_{y}—(CH₂—CH₂O)ₓ—H.

These polymers, also known under the designation of poloxamers or Pluronic, exhibit, on the one hand, a high biocompatibility and, on the other hand, on account of their strong tendency to form hydrogen bridges, a pronounced thermosensitivity. By selective copolymerization or block copolymerization from polyoxyethylene and polyoxypropylene, the critical phase transition temperatures can be adjusted in the range from 20 to 70° C. such that as the hydrophilic polyoxyethylene portion increases (normally to >50 mole percent), the phase transition can be shifted to temperatures >40° C. An alternative to manipulating the phase transition temperature is the addition of polyhydroxy compounds such as, for example, sorbite, sucrose or glycerol. These compounds shift the gel point to lower temperatures (<40° C.), whereas acids or salts such as, for example, NaCl, Na₂SO₃, Na₂SO₄, KC1 shift the phase transition to higher temperatures (>45° C.).

By combining polyoxyethylenes and polyoxypropylenes with diamines or triamines, such as, for example, ethylene diamine, a further group of thermosensitive, biocompatible polymers results which have the general formula:
[(R1R2) (R1R2)]=X═[(R1R2) (R1R2)],
which are also known under the designation of poloxamines. R1 and R2 represent a polyoxyethylene residue or a polyoxy-propylene residue, and X represents a polyfunctional amine. The syntheses of such copolymers are generally known from the state of the art.

The production of magnetic microparticles or nanoparticles could, surprisingly, be achieved by admixing up to 40% by volume of a water-based ferrofluid to the aqueous solutions of these polymers or copolymers. The mixtures are then suspended in an organic phase that is not miscible with the polymer phase—preferably oils having a viscosity of 40 to 120 cp—by adding 0.1 to 2 mole percent of a bifunctional or trifunctional crosslinking agent that is capable of crosslinking the terminal hydroxyl groups. Examples include: cyanuric chloride, diisocyanate, epichlorohydrin, dihalide, carbonyl diimidazole. Mechanical suspending of these mixtures may be accomplished optionally with a conventional stirrer or, to obtain nanoparticles, advantageously with a dispersing machine (e.g. T25 Ultraturrax, IKA, Germany), by applying a stirring speed >10,000 rpm. The volume ratio of the polymer phase to the suspension phase is generally 0.03 to 0.1.

Thermosensitive and biocompatible polymer carriers can, surprisingly, also be produced by suspending positively or negatively charged polymers, which have been dissolved in aqueous phases, in organic phases and by solidifying them, by subsequent addition of oppositely charged substances, to discrete spherical polymer particles. Examples of these are, without limiting the invention in any way: alginates, chitosan, nucleic acids, proteins and polyamino acids. To this end, the polymers are first transferred into a 1 to 10% aqueous solution. During subsequent suspension in a phase that is immiscible with water (e.g. vegetable or silicone oils or chlorinated hydrocarbons, ratio of polymer phase/continuous phase: 0.025-0.15), the dissolved polymers are crosslinked to spherical particles by adding oppositely charged substances. Examples of such substances with a crosslinking effect are bivalent salts, such as, for example, calcium chloride for alginates, nucleic acids and polyamino acids, or polyphosphates for chitosan.

To synthesize magnetic carriers, those water-based ferrofluids are added to the polymer solutions that are capable of forming stable, colloidally disperse solutions with the polymer phase.

Surprisingly, it is moreover also possible to use positively charged amines, such as spermine, spermidine and protamine, which occur in cells and enclose the DNA, to produce spherical magnetic particles. A preferred manner of synthesizing these particles starts out from a 0.5 to 10% nucleic acid buffer solution (pH >8.4). To the solution are added 10 to 40% by volume of a water-based ferrofluid, as well as, optionally, a water-soluble drug or bioactive substance. This mixture is suspended in a water-immiscible phase, preferably consisting of oils having a viscosity of 60 to 100 cp, with stirring. During the suspension process, 0.1 to 3 mole percent, relative to the content of nucleic acid, of the corresponding amines are added, which solidify the nucleic acid suspension to spherical particles. Depending on the conditions of stirring and on the concentrations of nucleic acid, particles having a size between 0.3 and 20 μm are obtained; generally the particle sizes are shifted to the nanometer range as the stirring speed is increased (>3000 rpm) and the concentration of nucleic acid is decreased (<5%). By addition of 0.1-5% by weight (relative to the nucleic acid portion) of a bioactive, preferably neutral, active agent to the nucleic acid solutions, drug carriers can be produced which are heated with an inductor coil (15 kA/m, 0.3 MHz, 4.4 kW) within 1 to 5 minutes so that within this heating-up period up to 70% of the encapsulated active substance is released on account of the partial swelling effect of the nucleic acid carrier.

Apart from magnet field-induced release of encapsulated drugs or bioactive substances, the media according to the present invention open up the possibility of coupling bioaffine ligands such as antibodies, cell receptors, anti-cell receptor antibodies, nucleic acids, oligosaccharides, lectins and antigens to the polymer carriers, with which the thermosensitive carriers can be an bound to certain target substances such as cells, biomolecules, viruses, bacteria or tissue compartments and/or selectively attached to these target organs according to the known principle of affinity. The polymer carriers can thus be attached specifically to T-cells, B-lymphocytes, monocytes, granulocytes, parent cells and leukocytes by coupling antibodies that are directed against the cell surface structures such as CD2, CD3, CD4, CD8, CD19, CD14, CD15, CD34 and CD45 (“cluster of differentiation”). Suitable media for realising the specific application by means of ligand-coupling polymer carriers are above all those media of the invention that contain functional groups in the form of carboxyl, hydroxyl or amino groups. Examples of these include polysaccharides, polyvinyl alcohol, gelatin, alginates and polylactides.

Coupling those antibodies or antibody fragments that are directed against a tumor cell antigen, initially creates the precondition for selectively concentrating the polymer carriers in the tumor tissue and attaching them to the tumor cells. Examples of such tumor markers and/or antigens, though these do not restrict the invention, include: tumor-associated transplantation antigen (TATA), oncofetal antigen, tumor-specific transplantation antigen (TSTA), p53-protein, carcinoembryonic antigen (CEA), melanoma antigens (MAGE-1, MAGE-B2, DAM-6, DAM-10), mucin (MUC1), human epidermis receptor (HER-2), alpha-fetoprotein (AFP), helicose antigen (HAGE), human papilloma virus (HPV-E7), caspase-8 (CASP-8), CD3, CD10, CD20, CD28, CD30, CD25, CD64, interleukin-2, interleukin-9, mamma-CA antigen, prostate-specific antigen (PSA), GD2 antigen, melanocortin receptor (MCIR), 138H11 antigen. The corresponding antibodies can optionally be used as monoclonal or polyclonal antibodies, as antibody fragments (Fab, F(ab′)₂), as single-chain molecules (scFv), as “diabodies”, “triabodies”, “minibodies” or bispecific antibodies.

For the parallel treatment of tumors, the tumor agents and cytostatic agents known from cancer therapy are encapsulated in the polymer particles. Examples of these include: methotrexate, cis-platinum, cyclophosphamide, chlorambucil, busulphan, fluorouracil, doxorubicin, ftorafur or conjugates of these substances with proteins, peptides, antibodies or antibody fragments. Conjugates of this type are known from the state of the art: “Monoclonal Antibodies and Cancer Therapy”, UCLA Symposia on Molecular and Cellular Biology, Reisfeld and Sell, Editors, Alan R. Riss, Inc., New York, 1985.

The known methods of coupling bioactive substances such as proteins, peptides, oligosaccharides or nucleic acids to solid carriers are used for the covalent binding of the bio- and affinity ligands or receptors to the polymer carrier. Coupling agents that are used here include, for example: tresyl chloride, tosyl chloride, cyanogen bromide, carbodiimide, epichlorohydrine, diisocyanate, diisothiocyanates, 2-fluoro-1-methylpyridinium-toluene-4-sulfonate, 1,14-butanediol-diglycidyl ether, N-hydroxysuccinimide, chlorocarbonate, isonitrile, hydrazide, glutaraldehyde, 1,1′,-carbonyl-diimidazole. Moreover, the bioligands can also be coupled with reactive heterobifunctional compounds that can enter into a chemical bond with both the functional groups of the matrix (carboxyl, hydroxyl, sulfhydryl, amino groups) as well as the bioligands. Examples in the sense of the invention are: Succinimidyl-4-(N-maleimido-methyl)-cyclohexane-1-carboxylate, 4-succinimidyloxycarbonyl-α-(2-pyridyldithio)toluene, succinimidyl-4-(p-maleimidophenyl)butyrate, N-γ-maleimido-butyryloxy succinimide, 3-(2-pyridyldithio)propionyl hydrazide, sulphosuccinimidyl-2-(p-azidosalicylamido)ethyl-1,3′-dithiopropionate. An expert in this field can use these coupling agents at any time in accordance with the information in G. T. Hermanson, “Bioconjugate Techniques”, Academic Press, San Diego, 1996.

The invention will be explained in more detail on the basis of the following descriptive but non-limiting examples. The particle sizes are determined by scattered light/laser diffraction using a Malvern MaserSizer 2000 (Malvern Instruments, Germany).

### EXAMPLE 1

2.8 g of polyvinyl alcohol, molar mass 204 kDa, are dissolved in 18 ml of ethylene glycol at 120° C. After the solution has cooled down to 80° C., 5 ml of a ferrofluid, stabilized with lauric acid, are added to the solution. This is followed by a five-minute-treatment in an ultrasonic bath. The dispersion is then left to cool down to 65° C. After dissolving 50 mg of Melphalan in the polymer phase, the mixture is suspended, while stirring (2000 rpm), in 100 ml of vegetable oil (viscosity 84 cp), which has been heated beforehand to 70° C. and contains 1.5% by volume of Pluronic 6200, 0.8% by volume of Dehymuls HRE and 2% by volume of Tween dissolved therein. During the suspension process the stirring vessel is cooled down with ice. After approx. 3 minutes the polymers precipitate as pearl-shaped particles. Stirring is continued for 15 minutes. Then, 100 ml of petroleum ether are added, and the magnetic particle fraction is separated using a handmagnet. This is followed by post-washing 10 times, alternately with petroleum ether and methanol. After drying under vacuum to a constant weight, magnet particles are obtained having a mean particle size of 12 μm.

These drug carriers can be used, inter alia, to treat carcinomas of the breast.

### EXAMPLE 2

500 mg of a triblock copolymer, prepared analogously to the specification from B. Jeong et al., Colloid Surfaces B Biointerfaces, Vol. 16, 185, 1999 and consisting of poly(ethylene glycol-lactide-ethylene glycol) (Mw: 11.8 kDa), are dissolved in 4 ml of 0.1 M Na-phosphate buffer, pH 7.4 and subsequently exposed to ultrasound for 5 min in an ultrasonic bath at 15° C. To this solution are added 100 mg of cobalt-ferrite powder (CoFe₂0₄, mean particle size 259 nm, which was prepared from CoCl₂ and FeCl₃. The dispersion is then exposed three times to ultrasound, using a high-performance ultrasound finger (from the firm of Dr. Hielscher, 80% amplitude) for 50 sec. under nitrogen atmosphere whilst being cooled with ice. This produces a stable colloid having a mean particle size of 645 nm.

### EXAMPLE 3

Magnetic chitosan nanoparticles are produced by ionic gelling of chitosan with Na-tripolyphosphate.

To 3.5 ml of an 0.6% chitosan-glutamate solution (Mw: 205 kDa) in bidistilled and degassed water, the pH of which has been adjusted to 5.5, are added 1.5 ml of an aqueous magnet-colloid solution (mean particle size 26 nm) which has been prepared according to Shinkai et al., Biocatalysis, Vol. 5, 61, 1991. To this dispersion are added in drops, using a pipette, 2.8 ml of a 0.084% Na-tripolyphosphate solution, containing 3 mg/ml of gonadotropin dissolved therein, whilst stirring vigorously (4500 rpm). After stirring for two minutes, the magnetic particles are placed in a glass column densely packed with steel wool (filling volume: approx. 4 ml; inside diameter: 0.5 cm) that is surrounded by a 3-cm-long, ring-shaped neodymium-boron-iron-magnet. The mixture is allowed to slowly (0.5 ml/min.) drip through the column. After this passage the column is rinsed ten times with approx. 20 ml of 30% ethanol. This is followed by washing five times with 0.1 M Na-phosphate buffer, pH 7.2, followed by washing ten times with bidistilled water. The magnetic polymer fraction on the column is then eluted with 10 ml of bidistilled water, after removing the magnet. The eluate obtained in this way is then freeze dried. Particles with a mean particle size of 672 nm are obtained. After appropriate dispersion in physiologic saline, the drug carriers can be used for hormone treatment.

### EXAMPLE 4

A 2.8% gelatin solution is prepared by heating to 90° C. in 1.5 ml of 0.05 M Na-phosphate buffer, pH 7.4. Then, the solution is brought down to 40° C., and initially 0.5 ml of ferrofluid EMG 507 (FerroTec, US) are added. The dispersion is then treated in the ultrasonic bath for 2 minutes at 40° C. 1 ml of 0.05 M Na-phosphate buffer solution, pH 7.4, which has been heated to 40° C. and which contains 0.25% of human insulin (Sigma) and 0.5% of polyvinyl alcohol (Mw: 22 kDa) dissolved therein, is added to the gelatin-ferrofluid dispersion. The resultant mixture is added to 80 ml of vegetable oil (viscosity 84 cp), which has been preheated to 40° C. and which contains 0.8% by volume of Pluronic L61, 0.8% by volume of Tetronic 1101 and 2.5% by volume of Dehymuls FCE dissolved therein, and this is homogenized for 2 minutes at 12,000 rpm, using a dispersing machine (T25 U1-traturrax, IKA, Germany), under an atmosphere of nitrogen. Then, the dispersion is cooled down to <10° C. by cooling with ice, which results in the precipitation of the gelatin particles. Following addition of 50 ml of petroleum ether, the magnetic fraction is separated by means of a neodymium-boron-iron handmagnet and post-washed 10 times, alternately with petroleum ether and ethanol. This is followed by washing 5 times with ice water. Polymer particles with a mean size of 1.4 μm are obtained.

### EXAMPLE 5

80 mg of a poly(lactide-coglycolide) (Mw 110 kDa), which has been prepared according to a specification from Zweers et al. (J. Biomed. Mater. Res. Part B, Vol. 66B, 559, 2002) and dried in a vacuum, are dissolved in 4 ml of acetone. 1.5 ml of the ferrofluid DKS1S21 (Liquids Research Ltd., Wales) and 8 mg of cyclophosphamide are added to the solution. The mixture is then dispersed for one minute at room temperature at 20,000 rpm, with the aid of a dispersing machine (T25 Ultraturrax, IKA, Germany), in 8 ml of water containing 60% by weight of MgCl₂ and 2% by weight of polyvinyl alcohol (Mw 22 kDA) as stabilizer. During the emulsifying process, 7.5 ml of water are added, and the stirring is continued for 20 seconds. This is followed by the high-gradient magnetic field separation analogous to Example 3. After the passage (0.5 ml/min), the column is rinsed ten times with approx. 20 ml of bidistilled water. After removing the magnet, the magnetic polymer carrier retained on the column is eluted three times, each time with 1.5 ml of physiologic saline. Nanoparticles having a mean particle size of 483 nm are obtained. Within 5 minutes, >50% of the encapsulated drugs, which can be used for tumor therapy, can be released with the aid of a high-frequency magnetic field (10 kA/m, 0.8 MHz, 4.8 kW).

### EXAMPLE 6

1.5 ml of magnetic colloid (2.2 mM Fe/ml, mean particle size 26 nm), which has been produced according to a specification from Shinkai et al., Biocatalysis, Vol. 5, 61, 1991, are mixed with 5 ml of a 0.05 M Na-carbonate buffer solution, pH 9.5, wherein 10% of the polyoxyethylene-polyoxypropylene copolymer (Pluronic® F68) are dissolved, and exposed to ultrasound for 5 minutes in an ultrasonic bath (500 W) whilst cooling with ice. Then, nitrogen is introduced into this mixture for 15 minutes at 20° C. To this dispersion is added 0.5 ml of 0.05 M Na-carbonate buffer solution, pH 9.5, which contains 1% by weight of somatotropin, 0.5% by weight of inosite und 0.05% by weight of human serum albumin dissolved therein. This mixture is treated with ultrasound for a further two minutes and then dispersed in 50 ml of sesame oil (viscosity 153 cp), which contains 2.5% by volume of Span 60 and 1.5% by volume of Dehymuls HRE7 dissolved therein, with stirring (1200 rpm) and introduction of nitrogen, at 20° C. During the dispersion process, 100 μl of divinyl sulfone are added using a pipette. Stirring of the mixture is continued for 2 hours. Separation and washing procedures are carried out analogously to Example 3. After freeze drying and dispersion in physiological saline, polymer carriers with a mean particle size of 0.767 μm are obtained.

Treating the particles in a magnetic alternating field (magnetic field: 10 kA/m; 0.6 MHz, coil diameter: 5.5 cm, 8 windings) triggers a deswelling process which releases more than 45% of the incorporated hormone within 5 minutes.

### EXAMPLE 7

Magnetic liposomes are produced according to the known processes. To this end, magnetite magnetic colloids (diameter approx. 15 nm) are produced and stabilised with lauric acid at 90° C. 0.18 ml of this colloid (61 mg Fe₃O₄/ml) are dialyzed for 72 h at 37° C. (Spectra/Por Dialysis Tubing, Spectrum medical Industries, Los Angeles, Calif., molar mass exclusion limit 12,000-14,000) with 9 ml of a vesicle dispersion, which has been obtained by ultrasound treatment, using an ultrasound finger (150 W), of a phospholipid-lidocaine mixture dimyristoyl phosphatidyl glycerin Na-salt/phosphatidyl ethanolamine-polyethylene glycol-biotin/lidocaine (phospholipid concentration: 8.4 uM/ml molar; ratio 9/1/0.5). The dialysis buffer (5 mM N-tris[hydroxymethyl]methyl-2-aminoethane sulfonic acid, TES, pH 7.0) is replaced after every 5 hours. Excess vesicles are separated by means of the column packed with steel wool (see Example 3). After separation, the magnetic fraction is rinsed several times with 4 ml of TES buffer. Then, the magnetic liposomes are obtained after removing the magnet by eluting three times, in each case with 2 ml of buffer solution. The molar ratio phospholipid/Fe₃O₄ is 0.69. In the subsequent treatment of the particles in a magnetic alternating field (magnetic field: 10 kA/m; 0.6 MHz, coil diameter: 5.5 cm, 8 windings) the vesicular structure of the liposomes is disintegrated within 5 minutes, und the encapsulated lidocaine is released completely. This drug carrier can be used as a local anesthetic.

### EXAMPLE 8

A cobalt-ferrite magnetic colloid (CoFe₂O₄) is produced from CoCl₂ and FeCl₃ and dispersed for 30 sec in water with the aid of a high-performance ultrasound finger (Dr. Hielscher, 80% amplitude) in the presence of 0.75% of polyacrylic acid (Mw: 5,500). 2 ml of the colloid (particle size 21 nm) containing 1.9 mM of Fe/ml are mixed with 5 ml of bidistilled and degassed water, wherein 5% by weight of isopropyl cellulose have been dissolved. The mixture is exposed to ultrasound for 10 minutes in an ultrasound bath at 20° C. Then, the mixture is dispersed in 70 ml of vegetable oil (viscosity 134 cp), which has been preheated to 70° C. and contains 1.5% of Tween 80, 2.5% of Pluronic PE 3100 and 2.5% of Span 85 dissolved therein, with the aid of a stirrer (1200 rpm). Stirring is continued for 10 minutes at that temperature. During this process, solid polymer particles are formed. After adding 100 ml of butanol, the magnetic fraction is separated by means of a handmagnet and is post-washed several times, alternately with petroleum ether and methanol. Magnetic particles with a mean particle size of 16 μm are obtained.

To 200 mg of the polymer particles thus produced are added 3 ml of 3.5 M NaOH and 5 ml of epichlorohydrine, and this is reacted for 2 h at 55° C. while stirring vigorously.

Thereafter, the magnetic particles are separated using a neodyme-iron-boron magnet. The product is suspended in approx. 10 ml of water and again magnetically separated. This washing/separation process is repeated 10 times, followed by washing once with acetone. The activated magnetic particle fraction is then converted with 2 ml of 0.1 M borate buffer, pH 11.4, which contains 10% of hexamethylene diamine, at 50° C. for 2 h. After magnetic separation, this is post-washed ten times with water. The product thus obtained is then brought to reaction with 2 ml of 0.1 M K-phosphate buffer, pH 7.0, which contains 12.5% of glutar aldehyde dissolved therein, for 2 h at 30° C. This is then post-washed for a period of 30 minutes, first 20 times with water and then five times with 0.1 M Na-phosphate buffer, pH 7.5. By three-hour incubation of 1.5 ml of 0.1 M Na-phosphate buffer, pH 7.5, which contains 0.2 mg of CD4 dissolved therein, polymer particles are obtained that can be utilized to bind HIV (human immunodeficiency virus). Through inductive heating of the virus-magnetic particle complex (4.8 kW, 0.5 MHz), temperatures (>60° C.) are achieved within ten minutes that are capable of killing the viruses.

1-27. (canceled)

28. Thermosensitive and biocompatible magnetic and/or metallic colloids-containing polymer particles that can be heated with the aid of a high-frequency magnetic alternating field and thereby undergo a change in their physical polymer structure, characterized in that the polymers used for producing the polymer particles according to the invention are selected from the group of the biocompatible polymers containing polyorthoesters, polyalkylcyanoacrylates, polyglycolic acid, polyether esters, polycarbonates, polyhydroxyalkanoates, polyhydroxy-acids, poly-(ε-caprolactone), polyamino-acids, polysaccharides, polysaccharide-(meth)acrylate derivatives, liposomes, isopropyl cellulose, hydroxyalkyl celluloses, cellulose acetate butyrate, starch, starch alkyl ethers, collagen, alginate, chitosan, polyvinyl alcohol, gelatin, ionically crosslinked multi-functional amines or linear copolymers, graft copolymers or block copolymers of these polymers, or dendrimers formed from said polymers.

29. The thermosensitive and biocompatible polymer particles according to claim 28, characterized in that said polymers consist of one or more copolymer(s) or block copolymer(s).

30. The thermosensitive and biocompatible, magnetic and/or metallic colloids-containing polymer particles according to claim 28, characterized in that said change in the physical structure consists in a swelling, deswelling, dissolving or gelation process or in a change in the geometric shape of the polymers.

31. The thermosensitive and biocompatible polymer particles according to claim 28, characterized in that they are spherical nanoparticulate or microparticulate particles or fibers, tubes or threads.

32. The thermosensitive and biocompatible polymer particles according to claim 30, characterized in that the change in geometric shape consists in a return to an original shape which the polymers possessed prior to a heat-induced change in shape (“shape-memory polymer”) .

33. The thermosensitive and biocompatible polymer particles according to claim 28, characterized in that the magnetic colloids are ferromagnetic, super paramagnetic or ferrimagnetic particles or ferrites or a ferrofluid with a particle size <1 μm and a Curie temperature in the range of from 30° C. to 100° C.

34. The thermosensitive and biocompatible polymer particles according to claim 28, characterized in that the metallic colloids consist of elements of the group 8, 9, 10 or 11 (group classification: new proposal by IUPAC 1986).

35. The thermosensitive and biocompatible polymer particles according to claim 28, characterized in that the magnetic and/or metallic colloids are stabilized with at least one surface-active substance that prevent(s) agglomeration of the colloids.

36. The thermosensitive and biocompatible polymer particles according to claim 35, characterized in that the surface-active substance(s) is/are alkyl and/or aryl groups-containing sulfonic acid, sulfonate or carboxylate derivatives or polyethylene glycol derivatives.

37. The thermosensitive and biocompatible polymer particles according to claim 28, characterized in that the polymers are crosslinked with a polyfunctional crosslinking agent selected from the group containing the dihalides, diisocyanates, diisothiocyanates, dioxiranes, dialdehydes and divinyl derivatives.

38. The thermosensitive and biocompatible polymer particles according to claim 37, characterized in that the crosslinking agent is present at a concentration of 0.05 to 10 mole percent, relative to the polymer.

39. The thermosensitive and biocompatible polymer particles according to claim 28, characterized in that the polymers contain reactive groups that couple to biomolecules.

40. The thermosensitive and biocompatible polymer particles according to claim 39, characterized in that the coupling groups are converted with peptides, proteins, antibodies, antigens, enzymes, cell receptor antibodies, antibodies to tumor markers, antibody fragments, artificially produced antibodies, modified antibodies, antibody conjugates, oligosaccharides, glycoproteins, lectins, nucleic acids, streptavidin or biotin.

41. The thermosensitive and biocompatible polymer particles according to claim 28, characterized in that active agents or drugs are encapsulated in the said polymers.

42. The thermosensitive and biocompatible polymer particles according to claim 41, characterized in that the encapsulated active agents or drugs are selected from the group of the hormones, cytostatic agents, antibodies, antibody derivatives, antibody fragments, cytokines, immunomodulators, antigens, proteins, peptides, lectins, glycoproteins, nucleic acids, antisense nucleic acids, oligosaccharides, antibiotics or generics.

43. A process for the production of thermosensitive and biocompatible polymer particles characterized in that the polymers are selected from the group of the biocompatible polymers containing polyorthoesters, polyalkylcyanoacrylates, polyglycolic acid, polyether esters, polycarbonates, polyhydroxyalkanoates, polyhydroxy-acids, poly-(ε-caprolactone), polyamino-acids, polysaccharides, polysaccharide-(meth)acrylate derivatives, liposomes, isopropyl cellulose, hydroxyalkyl celluloses, cellulose acetate butyrate, starch, starch alkyl ethers, collagen, alginate, chitosan, polyvinyl alcohol, gelatin, ionically crosslinked multi-functional amines or linear copolymers, graft copolymers or block copolymers of these polymers, or dendrimers formed from said polymers and that the polymers are heated by means of magnetic induction.

44. The process according to claim 43, characterized in that the polymers are produced by means of ring-opening polymerization, polycondensation, free-radical polymerization or ionic crosslinking.

45. The process according to claim 43, characterized in that the soluble polymer phase is dispersed, with stirring, in an organic phase that is immiscible with the polymer phase, and is solidified to form microparticulate or nanoparticulate particles by employing the suspension precipitation, the suspension crosslinking, the salting-out emulsification, or the solvent evaporation processes.

46. The process according to claim 43, characterized in that the magnetic and/or metallic colloids are added to the soluble polymer phase and after solidification of the polymer are present in the polymer matrix in colloidal disperse form.

47. The process according to claim 43, characterized in that ferromagnetic, superparamagnetic or ferrimagnetic substances or ferrites or ferrofluids with a particle size of <1 μm are used as magnetic colloids.

48. The process according to claim 43, characterized in that the concentration of the magnetic and/or metallic colloids added is 5 to 40% by weight, relative to the polymer phase.

49. The process according to claim 43, characterized in that mineral oils or vegetable oils having a viscosity of 40 to 400 cp, organic, water-immiscible solvents, or polyethylene glycols are used as organic phase.

50. The process according to claim 43, characterized in that in the organic phase there is/are dissolved 0.1 to 10% by volume of one or more surface-active substance(s) that are selected from the group of the alkyl sulfosuccinates, polyoxyethylene aryl ethers, polyoxyethylenes, polyoxyethylene sorbitan esters, polyoxyethylene adducts, polyethylene propylene oxide block copolymers, alkylphenoxypolyethoxyethanols, fatty alcohol polyethylene glycol ethers, polyglycerol esters, polyoxyethylene alcohols, polyoxyethylene sorbitan fatty acid esters, polyoxyethylene acids, or mixtures thereof.

51. The process according to claim 43, characterized in that peptides, proteins, antibodies, antigens, enzymes, cell receptor antibodies, antibodies to tumor markers, antibodies to tumor antigens, antibody fragments, artificially produced antibodies, modified antibodies, antibody conjugates, oligosaccharides, glycoproteins, lectins, nucleic acids, streptavidin or biotin are coupled to the polymers.

52. The process according to claim 43, characterized in that active agents or drugs are encapsulated in the polymer particles.

53. The process according to claim 43, characterized in that hormones, cytostatic agents, antibodies, cytokines, immunomodulators, antigens, proteins, peptides, lectins, glycoproteins, nucleic acids, antisense nucleic acids, oligosaccharides, antibiotics or generics are used as active agent.

54. The thermosensitive and biocompatible polymer particles according to claim 28 as a contrast-intensifying medium in NMR diagnostics, as carriers for active agents in medicinal therapy and diagnostics, as controllable carriers for reactants, as media for controlling microfluid processes, as separating medium in column chromatography, as media for setting and regulating pore sizes in membranes, as media for blocking blood vessels in tumor treatment, as artificial cell carriers, as separating medium for nucleic acids, cells, proteins, steroids, viruses or bacteria.
